(19) United States
(12) Patent Application Publication
(10) Pub. No.: US 2016/0016646 A1
(43) Pub. Date: Jan. 21, 2016

(54) OCEANIC NAVIGATION SYSTEM

(71) Applicant: DONGHATECH CO., LTD., Seo-gu Gwangju (KR)

(72) Inventors: Byoungdai Mun, Gwangju (KR); Taejin HA, Gwangju (KR); Byoungguk MIN, Gwangju (KR); Heeja JEONG, Gwangju (KR); Jeongmin PARK, Gwangju (KR)

(73) Assignee: Dae Moon Information Co., Ltd., Gwangju (KR)

(21) Appl. No.: 14/775,250

(22) PCT Filed: Aug. 22, 2013
(86) PCT No.: PCT/KR2013/007515

§ 371 Date: Sep. 11, 2015

(30) Foreign Application Priority Data
Mar. 11, 2013 (KR) 10-2013-0025818

(51) Int. Cl. B63B 49/00; G01P 5/02; G01S 19/42; G01N 33/18; G01C 21/00; G01C 21/20

(52) U.S. Cl. CPC B63B 49/00; G01C 21/005; G01C 21/20; G01S 19/42; G01N 33/18; G01P 5/02

(57) ABSTRACT

The present invention relates to an oceanic navigation system, and more particularly to an oceanic navigation system including an oceanic navigation control apparatus which is interfaced with a GPS reception part and various sensing devices so as to check the problems of a vessel in real time, and a smart terminal for executing an oceanic navigation applet so as to display the state information and the navigation information provided by the oceanic navigation control apparatus on the display screen, thus improving the portability thereof.

### TECHNICAL FIELD

The present invention relates to an oceanic navigation system, and more particularly, to an oceanic navigation system which includes an oceanic navigation control apparatus, and is capable of checking problems within a ship in real time by interfacing with a GPS reception part and various sensing devices and improving portability by executing state information and navigation information provided from the oceanic navigation control apparatus through a smart terminal in an oceanic navigation application to output the executed information on a display screen.

### BACKGROUND ART

In recent years, as traffic equipment is developed in the downtown area and even in the coastal area, good-quality articles can be provided to consumers of the island area in the coastal area due to traffic culture of a one-day life zone from a production area to a consumer.

Accordingly, as the fishing industry such as high value-added fishing nets and fish farms and the number of people who do marine leisure sports activities, that is, the number of people who experience marine culture such as sea fishing and island tour increase, there is an increasing trend toward an increase in the number of passenger boats or small owner-driven ships as marine transportation means of the island area.

Thus, as the number of people who use the marine transportation means due to the development of marine leisure sports culture and the operation model of the fishing industry increases, in order to satisfy the requirements of a ship operator operated by the younger generation, there is an increasing need for the development of a terminal and an operating program thereof so as to provide a service of an electronic navigation chart database (DB) including various oceanic information items and contents.

Particularly, in the area of the marine leisure sports activities using the owner-driven ship or the small ship, the content of the ocean guide, put in another way, the electronic navigation chart and the operating program thereof allow the ship to avoid the danger area such as fishing farms, fishing nets or rocks.

When the electronic navigation chart and the operating program thereof guide a destination on the ocean so as to prevent the ship from losing direction and meeting with distress in the foggy area, there is an increasing need for various oceanic information contents and electronic navigation chart databases in order to provide services such as a guide to route deviation in addition to guiding a detour around the danger area.

In the electronic navigation chart of the related art, only longitude and latitude coordinates are displayed on the screen of the terminal, simple electronic navigation chart data is merely provided so as to allow the ship operator to directly see the data and operate the ship, like a navigation chart made of pure paper.

In the case of the oceanic navigation of the related art, since data is selectively provided to a user through wireless communication by using only a server all the time, when communication is disconnected between the operator and the server, it is not able to detect the position coordinate of the ship, and it is not able to provide a ship voyage guide service using the entire ocean route and optimum ship's route network data or route guide of searching for a detour around the danger area with screen and sound.

When the oceanic navigation of the related art is applied to the small ship or the general ship, there are various limitations that it is difficult for the operator to easily attach or detach the oceanic navigation to the small ship or the general ship and it is not able to provide the ship route search and navigation guide information using the ship's route network attribute optimized for ship characteristics so as to allow the small ship or the general ship to sail.

The oceanic navigation of the related art does not interface with devices provided within the existing ship, is not able to be in line with an image system, and is not able to a collision avoidance and collision prediction warning system.

### DISCLOSURE

### Technical Problem

A first object of the present invention is to provide an oceanic navigation system which includes an oceanic navigation control apparatus, and is capable of checking problems within a ship in real time by interfacing with a GPS reception part and various sensing devices and improving portability by executing state information and navigation information provided from the oceanic navigation control apparatus through a smart terminal in an oceanic navigation application to output the executed information on a display screen.

A second object of the present invention is to provide an oceanic navigation system capable of previously recognizing the collision with the surrounding ship or obstacle by analyzing an image frame obtained from a camera part that obtains surrounding image information to recognize an object appearing near a virtual boundary line, and analyzing the movement direction of the object to check whether or not an abnormal situation occurs.

A third object of the present invention is to provide an oceanic navigation system which includes an auxiliary magnetic compass that measures an azimuth, and is capable of obtaining azimuth information from the magnetic compass and displaying the obtained azimuth information when it is not able to receive the azimuth from the GPS reception part.

A fourth object of the present invention is to provide an oceanic navigation system which provides a submarine topographical map, and is capable of preventing an accident that causes a big accident due to the collision of the ship with a submarine obstacle at unawares by allowing an operator to check a distance between a current position and a submarine topography.

### Technical Solution

The technical solution of the present invention for achieving the above objects is suggested.

An oceanic navigation system of the present invention includes an orientation instrument that obtains azimuth information; a sounding device that measures a water level; a water temperature measurement device that measures a water temperature; a speedometer that measures a speed; an anemometer that measures a wind speed and a wind direction; a lighting device; an oceanic navigation control apparatus; and a smart terminal that executes state information and navigation information provided from the oceanic navigation control apparatus in an oceanic navigation application and displays the provided information.

### Effect of the Invention

It is possible to improve portability and provide extensibility so as to be used for any smart terminal by including an oceanic navigation control apparatus and being capable of checking problems within a ship in real time by interfacing with a GPS reception part and various sensing devices and improving portability by executing state information and navigation information provided from the oceanic navigation control apparatus through a smart terminal in an oceanic navigation application to output the executed information on a display screen.

It is possible to provide an effect capable of previously recognizing the collision with the surrounding ship or obstacle by analyzing an image frame obtained from a camera part that obtains surrounding image information to recognize an object appearing near a virtual boundary line, and analyzing the movement direction of the object to check whether or not the abnormal situation occurs.

It is possible to prevent anxiety about the navigation when it is not able to chase a position by including an auxiliary magnetic compass that measures an azimuth and being capable of obtaining azimuth information from the magnetic compass and displaying the obtained azimuth information when it is not able to receive the azimuth from the GPS reception part.

It is possible to an effect capable of providing a submarine topographical map, and preventing an accident that causes a big accident due to the collision of the ship with a submarine obstacle at unawares by allowing an operator to check a distance between a current position and a submarine topography.

### DESCRIPTION OF DRAWINGS

FIG. 1 is a diagram showing the entire configuration of an oceanic navigation system according to an embodiment of the present invention.

FIG. 2 is a block diagram of an oceanic navigation control apparatus of the oceanic navigation system according to the embodiment of the present invention.

FIG. 3 is a block diagram of central control means of the oceanic navigation system according to the embodiment of the present invention.

FIG. 4 is an exemplary screen output on a smart terminal of the oceanic navigation system according to the embodiment of the present invention.

### BEST MODE

The best mode for implementing the invention is as follows.

An oceanic navigation system of the present invention includes an orientation instrument 200 that obtains azimuth information; a sounding device 300 that measures a water level; a water temperature measurement device 400 that measures a water temperature; a speedometer 500 that measures a speed; an anemometer 600 that measures a wind speed and a wind direction; a lighting device 700; an oceanic navigation control apparatus 100; and a smart terminal 1000 that executes state information and navigation information provided from the oceanic navigation control apparatus in an oceanic navigation application and displays the provided information.

The oceanic navigation control apparatus 100 includes:

a GPS reception part 105 that receives ship positional information of an operator from a GPS satellite;

an electronic navigation chart DB 110 that stores a background image of an ocean, text name data and route network data;

a key input part 115 that generates a user operation signal to the operator to allow the operator to select a guide mode of an electronic navigation chart;

a fuel amount measuring part 120 that measures a fuel amount;

a lighting sensing part 125 that senses whether or not the lighting device is broken down;

a measurement interface part 130 that interfaces with the orientation instrument, the sounding device, the water temperature measurement device, the speedometer, the anemometer and the lighting device to receive the measurement information, provides the received information to central control means, and obtains a control signal of a lighthouse lantern or a flashing device from the central control means;

a measurement data obtaining part 135 that obtains the measurement data from the orientation instrument, the sounding device, the water temperature measurement device, the speedometer and the anemometer that interface with the measurement interface part;

an oceanic weather information obtaining part 140 that obtains oceanic weather information by referring to the ship positional information of the operator received by the GPS reception part;

an electronic navigation chart reading module 151 that reads data matching the guide mode of the electronic navigation chart selected by the operator through the key input part or the ship positional information of the operator received by the GPS reception part from the electronic navigation chart DB;

a first screen output controlling module 152 that controls such that the read data is executed by an operating program and the executed data is displayed on a screen;

a second screen output controlling module 153 that controls such that information regarding an azimuth, a water level, a water temperature, a speed, a wind direction and a wind speed obtained by the measurement data obtaining part and position-based oceanic weather information obtained by the oceanic weather information obtaining part are obtained and the obtained information is displayed on a screen;

central control means 150 that includes a danger analyzing module 154 which obtains information regarding sudden stop and transmission of a ship to analyze whether or not the ship is abnormal and whether or not the ship meets with an accident; and

a ship information DB 160 that stores the state information and the navigation information obtained by the measurement data obtaining part.

### DESCRIPTION OF THE PREFERRED EMBODIMENTS

The technical solution described above will be described in more detail with reference to the accompanying drawings so as to be implemented by those skilled in the art.

The terms used in the present invention will be described as follows.

A general electronic navigational chart (ENC) is a navigation chart which is created by a national hydrographic and oceanographic office administration and includes numerical data contents obtained by standardizing the structural type of ocean-related information, that is, land and island areas, a lighthouse, a marina, a quay, a port, a rock, a route, a turning point, depth of ocean, an anchorage, a sunken vessel, a fishing farm, a fishery zone, a buoy, a submarine cable, and a danger mark into a digital type. The ENC called an electronic navigational chart is a navigation chart required to electronically display positional information during sailing of a ship or flight of an airline.

The route network data refers to a ship's route in which structured editing has been performed and the attribute has been input so as to allow the ship to sail on the ocean, and is data converted into service data by newly generating a route in a safe area separated from the oceanic obstacle area by a predetermined distance (about 50 m or more) and inputting a ship's route network attribute to the generated route so as to allow the ship to avoid an oceanic obstacle area such as fishing farms, rocks or fishing nets.

Ocean depth data means information constructed by building a database of depth information of the ocean using a numerical value and coordinate system, and is data converted into service data by being categorized as surface-type polygon data and line-type polyline data and inputting the data so as to express submarine topography and a slope with respect to the depth of the ocean.

The danger area on the ocean means an area obtained by being categorized as a fishery area such as a fishing farm or a fishing net, a rock, and a low depth, inputting attribute to the categorized items, and being categorized as danger area data for warning for a detour around an oceanic obstacle when the general ships sail or so as to be applied to a detour searching algorithm.

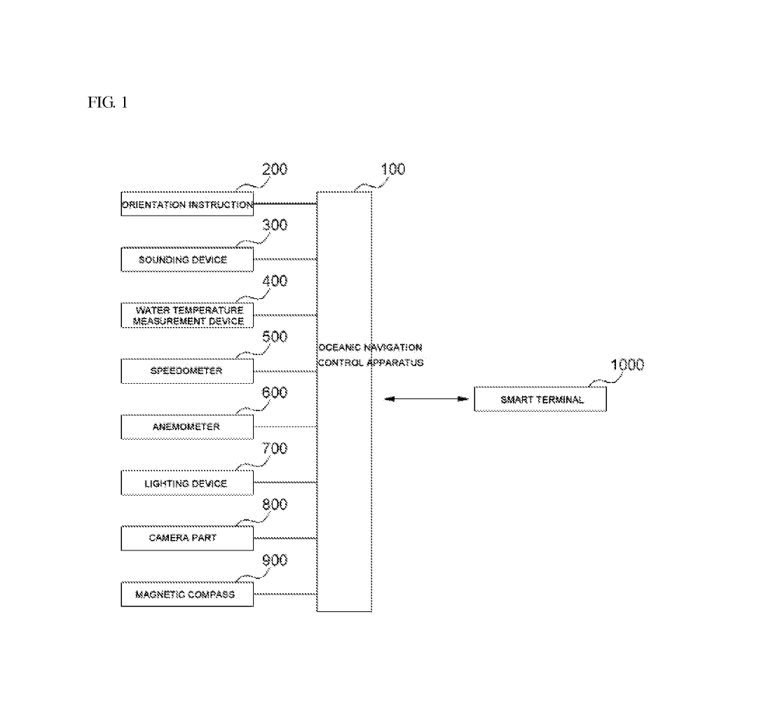

FIG. 1 is a diagram showing the entire configuration of an oceanic navigation system according to an embodiment of the present invention.

As shown in FIG. 1, the oceanic navigation system includes an orientation instrument 200 that obtains azimuth information; a sounding device 300 that measures a water level; a water temperature measurement device 400 that measures a water temperature; a speedometer 500 that measures a speed; an anemometer 600 that measures a wind speed and a wind direction; a lighting device 700; an oceanic navigation control apparatus 100; and a smart terminal 1000 that executes state information and navigation information provided from the oceanic navigation control apparatus in an oceanic navigation application and displays the provided information.

That is, various information items related to the navigation are provided on a display screen of the smart terminal. The oceanic navigation control apparatus obtains an azimuth, a water level, a water temperature, a speed, a wind direction, a wind speed, and whether or not the lighting is turned on from the measurement devices, processes the obtained information, and provides the processed information to the start terminal so as to display the processed information on the screen.

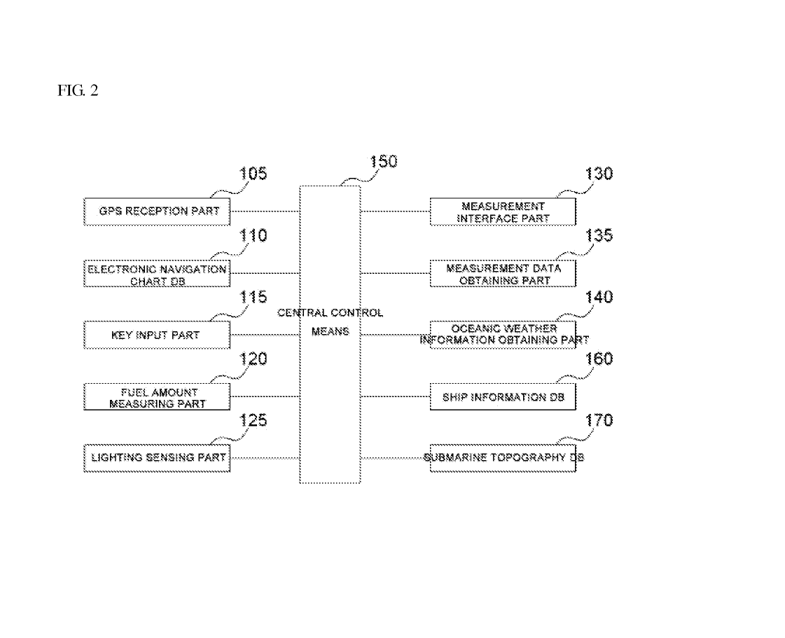

FIG. 2 is a block diagram of the oceanic navigation control apparatus of the oceanic navigation system according to the embodiment of the present invention.

That is, the oceanic navigation control apparatus 100 includes:

GPS reception part 105 that receives ship positional information of an operator from a GPS satellite;

an electronic navigation chart DB 110 that stores a background image of an ocean, text name data and route network data;

a key input part 115 that generates a user operation signal to the operator to allow the operator to select a guide mode of an electronic navigation chart;

a fuel amount measuring part 120 that measures a fuel amount;

a lighting sensing part 125 that senses whether or not the lighting device (lighthouse lantern or flashing device) is broken down;

a measurement interface part 130 that interfaces with the orientation instrument, the sounding device, the water temperature measurement device, the speedometer, the anemometer and the lighting device to receive the measurement information, provides the received information to central control means, and obtains a control signal of the lighthouse lantern or the flashing device from the central control means;

a measurement data obtaining part 135 that obtains the measurement data from the orientation instrument, the sounding device, the water temperature measurement device, the speedometer and the anemometer that interface with the measurement interface part;

an oceanic weather information obtaining part 140 that obtains oceanic weather information by referring to the ship positional information of the operator received by the GPS reception part;

an electronic navigation chart reading module 151 that reads data matching the guide mode of the electronic navigation chart selected by the operator through the key input part or the ship positional information of the operator received by the GPS reception part from the electronic navigation chart DB;

a first screen output controlling module 152 that controls such that the read data is executed by an operating program and the executed data is displayed on a screen;

a second screen output controlling module 153 that controls such that information regarding an azimuth, a water level, a water temperature, a speed, a wind direction and a wind speed obtained by the measurement data obtaining part and position-based oceanic weather information obtained by the oceanic weather information obtaining part are obtained and the obtained information is displayed on the screen;

central control means 150 that includes a danger analyzing module 154 which obtains information regarding sudden stop and transmission of a ship to analyze whether or not the ship is abnormal and whether or not the ship meets with an accident; and

a ship information DB 160 that stores the state information and the navigation information obtained by the measurement data obtaining part.

An ultrasonic anemometer based on NMEA 2000 is applicable to the anemometer.

The wind speed may be obtained from a time at which pulses of two pairs of transmission and reception ultrasonic sensors that are provided in east, west, south and north directions reach.

The pulses are sent from the transmission sensors, a timer is operated from a time at which the pulses are transmit, and a time is calculated using a timer count value until the pulses are received by the reception sensors.

Times at wind speeds in four orientations (E->W, W->E, S->N, N->S) are measured, and wind direction vectors are extracted using four wind speeds.

The ultrasonic anemometer is a technology known to those skilled in the art, and thus, the detailed description of an operation principle will be omitted.

In this case, after the wind direction and the wind speed are obtained, data regarding the wind direction and the wind speed is transmitted on a NMEA 2000 network.

In order to access the NMEA 2000 network, the ultrasonic anemometer needs to respond to an address request of the NMEA 2000 network.

To achieve this, a source address needs to be included, and a data packet of a self-ID PGN 60928 needs to be included. The data regarding the wind speed and the wind direction needs to include an address request PGN, a request PGN, and a multi-packet.

Next, the configuration of the oceanic navigation control apparatus will be described in detail.

That is, the GPS reception part 105 receives the ship positional information of the operator from the GPS satellite.

That is, the GPS reception part includes a reception circuit and a sensor that measure time information, longitude and latitude coordinates, altitude information, a movement direction, speed information, and the arrangement state and signal strength of the GPS satellites.

The electronic navigation chart DB 110 stores the background image of the ocean, the text name data and the route network data.

That is, voice and sound guide data that manage route guide and warning sound files with voice and sound, icon and symbol data, a general GUI design menu image data from an initial menu to sub-menus, an electronic navigation chart DB operator, the electronic navigation chart DB, and an operation execution program for executing and driving them are generally embedded.

In general, the electronic navigation chart refers to a service data format of an electronic navigation chart that includes a route network layer for realizing voyage route search and navigation guide function using a route network and a ship's route network obtained by generating a ship's route in which the ship can sail and assigning attribute information to the generated ship's route, an ocean background image layer constructed by generating the information regarding the ocean depth, the fishing farms and the fishing nets and the navigation in a polygon shape and a polyline shape and using the information regarding the ocean depth, the fishing farm mark and the route on the ocean as a numerical value map data, a land area layer capable of detecting the Gulf of the land, the shore of the peninsula, and the land such as an island of the island area and determining the departure, entrance or route course for a detour of the ship, an ocean danger area layer capable of performing a detour around the obstacle and danger area of the ship voyage, automatically searching for the route in advance and guiding so as to allow the ship to sail in the route by automatically detecting the rock of the ocean, the rock that is not seen under the ocean, that is, the rock area, the fishery area such as the fishing farm or the fishing net, the anchorage, and the sunken vessel area in advance with a predetermined distance, and an ocean POI searching layer such as a lighthouse, a marina, a quay, a port, a passenger terminal, island tour information, or a fishing point for helping the operator during voyage guide.

Here, in order for the operator to manually or automatically select the guide mode of the electronic navigation chart using the key input part, the electronic navigation chart database has a structure in which a certain area and an area having a low water depth are organized into a safe area by being separated toward the ocean from the land or the shore of the island by a predetermined distance, the structured editing is performed and the attribute information is input in order to previously build a database of summary information such as a course name, a necessary time, and a section distance for each passenger ship route and to generate a ship's route in which only the small ship sails.

In order for the operator to operate the ship so as to avoid the area of the ocean obstacle such as the fishing farm, the rock or the fishing net, a route is constructed as a ship's route safe area network DB by being separated from the area of the ocean obstacle by a predetermined distance, and the operator can recognize a navigation plan together with the summary information of the route course on the screen of the terminal by using the ship's route safe area network DB as route network data.

A background image of domestic shore and route network data based on Google to which the electronic navigation chart DB is applied or an open API map of NAVER Corporation may be used in the present invention.

That is, general people can easily see by mapping the important information (raw data) of the electronic navigation chart with the coordinate, and since 3G communication is available in adjacent seas of our country, the electronic navigation chart is actively utilized.

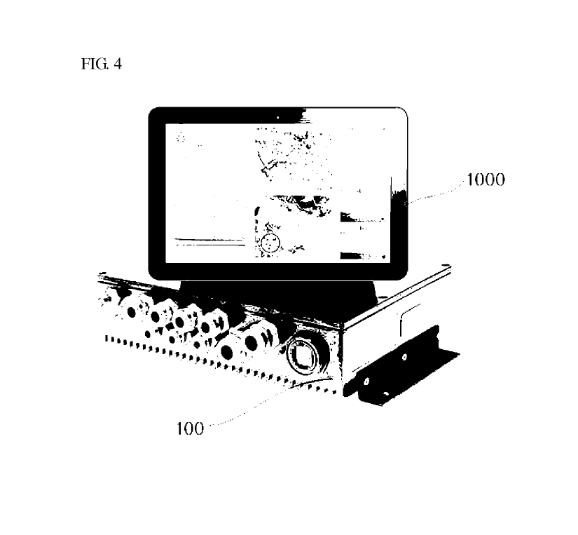

As shown in FIG. 4, the fuel amount measuring part 120 measures the fuel amount within the ship, and displays a current fuel amount on the screen of the smart terminal.

For example, the lighting sensing unit senses whether or not breakdown occurs by checking the state of the lighting device, that is, the lighthouse lantern or the flashing device, and outputs the current state on the screen of the smart terminal. Thus, it is possible to previously prevent the accident when the lighting is broken down during night voyage.

To achieve this, the measurement interface part 130 interfaces with the orientation instrument, the sounding device, the water temperature measurement device, the speedometer, the anemometer and the lighting device to receive the measurement information, provides the received information to central control means, and obtains a control signal of the lighthouse lantern or the flashing device from the central control means.

The lighthouse lantern or the flashing device can be turned on or off by the smart terminal depending on the control signal of the lighthouse lantern or the flashing device.

For example, the NMEA 2000 network is constructed, and the GPS, the sounding device, the water temperature measurement device, the speedometer, and the anemometer are connected.

Specifically, these devices are connected to the NMEA 2000 through a CAN port of FPGA, and are connected to NMEA 0183 through a UART port.

Information of the measurement devices connected to the NMEA 2000 network can be obtained in addition to engine data and fuel tank data generated in the FPGA.

The NMEA 2000 protocol has been started by NMEA of United State since 1994 in cooperation with U.S. Coast Guard, universities, voyage communication equipment companies all over the world, and CAN solution companies.

The NMEA 2000 protocol of which the standard has been initially completed on September 2001 is a real-time communication network capable of mutually connecting the electronic devices for the ship and performing full-duplex multiplexing transmission and reception at low cost.

IMO defines the NMEA 2000 protocol as a standard of an instrument network, and IEC defines the NMEA 2000 protocol as a national standard by means of 61162-3.

ISO (International Organization for Standardization) defines the NMEA 2000 protocol as a standard network of a SOLAS ship.

Unlike an existing NMEA 0183 protocol of which a transmission speed is 4,800 bps or 38,400 bps, since the NMEA 2000 protocol maintains a transmission speed of 250 kbps, it is possible to ensure a high transmission speed, it is possible to perform full-duplex multiplexing transmission and reception, and it is not necessary to provide a separate server.

When the ship is connected to a normally operated network, even though partial equipment is broken down, the entire network is not affected.

Due to such a feature, in the field of ship materials, the NMEA 2000 protocol is adopted, and products using the NMEA 200 protocol are commercially used. In the present invention, the NMEA 200 protocol is applied in order to interface the measurement data.

In this case, the measurement data obtaining part 135 obtains the measurement data from the orientation instrument, the sounding device, the water temperature measurement device, the speedometer and the anemometer that are interfaced by the measurement interface part, and provides the obtained measurement data to the central control means.

Meanwhile, in the present invention, an oceanic weather information obtaining part 140 is provided, and is configured to obtain oceanic weather information by referring to the ship positional information of the operator received by the GPS reception part.

That is, the current wind speed, wind direction, orientation, temperature, water temperature, altitude and coordinate are provided on the screen. However, the oceanic weather information is obtained in order to determine whether the operator operates the ship up to a desired destination or returns the ship to its departure in real time by checking the weather state near adjacent seas with reference to the ship positional information.

In the adjacent seas, since the 3G communication is available, the oceanic weather information can be obtained from the National Weather Service or Korea Coast Guard using the 3G communication.

The state information and the navigation information obtained by the measurement data obtaining part are stored and managed in the ship information DB 160.

In so doing, the state information and the navigation information are utilized as base data when an emergency situation occurs.

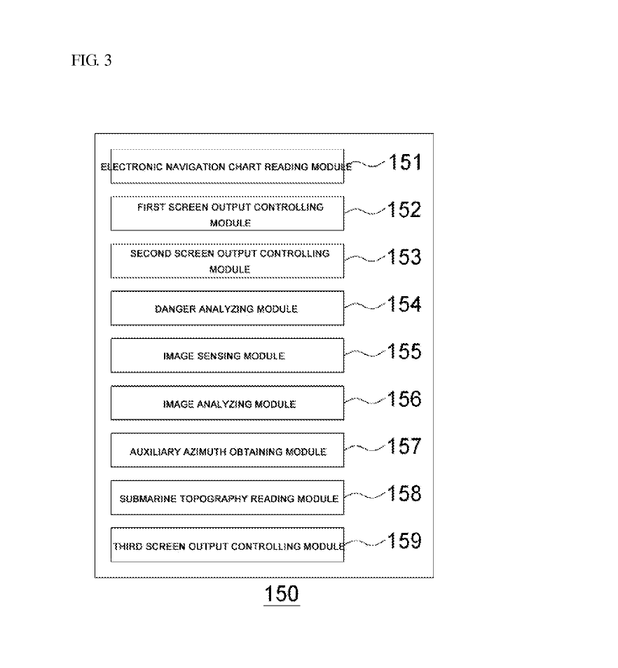

FIG. 3 is a block diagram of the central control means of the oceanic navigation system according to the embodiment of the present invention.

That is, the central control means 150 includes:

the electronic navigation chart reading module 151 that reads the data matching the guide mode of the electronic navigation chart selected by the operator through the key input part or the ship positional information of the operator received by the GPS reception part from the electronic navigation chart DB;

the first screen output controlling module 152 that controls such that the read data is executed using the operating program and the executed data is displayed on a screen;

the second screen output controlling module 153 that controls such that the information regarding the azimuth, the water level, the water temperature, the speed, the wind direction and the wind speed obtained by the measurement data obtaining part and the position-based oceanic weather information obtained by the oceanic weather information obtaining part are obtained and the obtained information is displayed on the screen; and

the danger analyzing module 154 that obtains the sudden stop and transmission of the ship to analyze whether or not the ship is abnormal and whether or not the ship meets with an accident.

The electronic navigation chart reading module 151 reads the data matching the guide mode of the electronic navigation chart selected by the operator through the key input part or the ship positional information of the operator received by the GPS reception part from the electronic navigation chart DB. In this case, background data of the corresponding ocean and a navigation network layer corresponding to a current position of the operator are read from the electronic navigation chart DB, and navigation is guided with sound and screen by referring to a destination designated by the key input part of the operator and a current position of the operator.

In this case, the first screen output controlling module 152 controls such that the read data is executed by the operating program and the executed data is displayed on the screen.

As shown in FIG. 4, the second screen output controlling module 153 controls such that the information regarding the azimuth, the water level, the water temperature, the speed, the wind direction and the wind speed obtained by the measurement data obtaining part and the position-based oceanic weather information obtained by the oceanic weather information obtaining part are obtained and the obtained information is displayed.

That is, the first screen output controlling module outputs a route and a surrounding situation on the screen, and the second screen output controlling module allows the operator to check the current state of the ship by outputting the various measured information items and the obtained weather information in a predetermined area in real time.

Meanwhile, the central control means further includes a danger analyzing module 154 that obtains the information regarding the sudden stop and transmission of the ship, and analyzes whether or not the ship is abnormal and whether or not the ship meets with an accident.

Put in another way, whether or not the ship is abnormal and whether or not the ship meets with an accident are analyzed by analyzing data such as the transmission and sudden stop of the ship and the number of times the ship alters the route.

In general, for example, it is determined whether or not the ship totters or sails zigzag. Whether or not sudden deceleration and acceleration are performed is determined in such a manner that it is determined as sudden deceleration when speed differences are added up for all track sections during a predetermined time interval and an average speed for each track section is suddenly decreased, whereas it is determined as sudden stop by determining a moderating ratio per minute with a predetermined calculation time interval of less than 5 minutes.

The technology of determining the transmission and sudden stop is a technology known to a person having ordinary skill in the art, and should be sufficiently understood by the above description.

In addition, as shown in FIG. 4, a camera part 800 that obtains surrounding image information may be further included.

In this case, the central control means 150 further includes an image monitoring module 155 that generates a virtual boundary line on a surrounding image collected by the camera part and recognizes an object appearing near the virtual boundary line, and an image analyzing module 156 that analyzes the movement direction of the object recognized by the image monitoring module and recognizes whether or not the ship is abnormal.

That is, the image monitoring module 155 can generate the virtual boundary line on the image collected by the camera part, and can recognize the object appearing near the virtual boundary line.

In this case, the virtual boundary line means an external boundary line along a distance from the ship, and the image analyzing module determines that the ship is abnormal when the object moves to the inside of the virtual boundary line, and analyzes whether or not another ship or obstacle approaches the ship.

In such a case, the central control means may display the positions of the camera parts provided on the screen on which the shape of the ship is drawn, may display the abnormal state on the camera part when the ship is abnormal, and may output the window of the screen when the operator touches the displayed abnormal state.

As shown in FIG. 4, areas may be designated in any positions on the screen, and the image information items obtained by the camera parts may be displayed on the designated areas.

The information items may be stored in the ship information DB, and the abnormal state may be informed to an ocean rescue team so as to take rapid follow-up measures.

The system of the present invention may further include:

a magnetic compass 900 that measure an azimuth.

In this case, the central control means 150 may further include an auxiliary azimuth obtaining module 157 that obtains azimuth information from the magnetic compass when it is not able to receive the azimuth information from the GPS reception part.

In other words, the orientation information is provided by including the magnetic compass as safe auxiliary means and obtaining the measurement data from the magnetic compass.

When it is not able to receive the azimuth information from the GPS reception part, since it is difficult to track the current coordinate, the operator is obviously flustered.

Thus, the safe auxiliary means is provided, and only the azimuth information is expressed.

Since an orientation signal is the most important data in the navigation of the ship, a separate area is designated, and the current azimuth information is displayed as shown in FIG. 4.

The oceanic navigation control apparatus 100 further includes:

a submarine topography DB 170 that stores submarine topography data, as an additional configuration.

That is, the submarine topography data is provided. In this case, the central control means 150 further includes:

a submarine topography reading module 158 that reads submarine topography data matching a guide mode of the electronic navigation chart selected by the operator through the key input part or the ship positional information of the operator received from the GPS reception part from the submarine topography DB; and

a third screen output controlling module 159 that controls such that the ship positional information of the operator received by the GPS reception part maps with the read submarine topography data and the mapped data is displayed on the screen.

Specifically, the submarine topography reading module 158 reads the submarine topography data matching the guide mode of the electronic navigation chart selected by the operator through the key input part or the ship positional information of the operator received by the GPS reception part.

That is, the height of the ocean floor can be graphically checked in real time by referring to the current positional information, and can checked by the operator through the screen of the ocean floor in the guide route up to the destination. Thus, it is possible to previously recognize the collision that the operator might have had.

To achieve this, the third screen output controlling module 159 controls such that the ship positional information of the operator received by the GPS reception part maps with the read submarine topography data and the mapped data is displayed on the screen.

Accordingly, it is possible to an effect capable of providing a submarine topographical map, and preventing an accident that causes a big accident due to the collision of the ship with a submarine obstacle at unawares by allowing an operator to check a distance between a current position and a submarine topography.

As a result, according to the present invention, it is possible to improve portability and provide extensibility so as to be used for any smart terminal by including an oceanic navigation control apparatus and being capable of checking problems within a ship in real time by interfacing with a GPS reception part and various sensing devices and improving portability by executing state information and navigation information provided from the oceanic navigation control apparatus through a smart terminal in an oceanic navigation application to output the executed information on a display screen.

It is possible to provide an effect capable of previously recognizing the collision with the surrounding ship or obstacle by analyzing an image frame obtained from a camera part that obtains surrounding image information to recognize an object appearing near a virtual boundary line, and analyzing the movement direction of the object to check whether or not the abnormal situation occurs.

Although the present invention has been described in connection with the aforementioned embodiment, the present invention may be variously implemented without departing from the technical spirit of the present invention, and the various implementations should be included in the present invention.

### INDUSTRIAL APPLICABILITY

The present invention is applied to the ship-related industry, and thus, it is possible to previously recognize the collision of the ship with another surrounding ship or obstacle.

1. An oceanic navigation system comprising:
an orientation instrument (200) that obtains azimuth information;
a sounding device (300) that measures a water level;
a water temperature measurement device (400) that measures a water temperature;
a speedometer (500) that measures a speed;
an anemometer (600) that measures a wind speed and a wind direction;
a lighting device (700);
an oceanic navigation control apparatus (100); and
a smart terminal (1000) that executes state information and navigation information provided from the oceanic navigation control apparatus (100) in an oceanic navigation application, and outputs the provided information,
wherein the oceanic navigation control apparatus (100) includes
a GPS reception part (105) that receives ship positional information of an operator from a GPS satellite,
an electronic navigation chart DB (110) that stores a background image of an ocean, text name data and route network data,
a key input part (115) that generates a user operation signal to the operator to allow the operator to select a guide mode of an electronic navigation chart,
a fuel amount measuring part (120) that measures a fuel amount,
a lighting sensing part (125) that senses whether or not the lighting device is broken down,
a measurement interface part (130) that interfaces with the orientation instrument, the sounding device, the water temperature measurement device, the speedometer, the anemometer and the lighting device to receive the measurement information, provides the received information to central control means, and obtains a control signal of a lighthouse lantern or a flashing device from the central control means,
a measurement data obtaining part (135) that obtains the measurement data from the orientation instrument, the sounding device, the water temperature measurement device, the speedometer and the anemometer that interface with the measurement interface part,
an oceanic weather information obtaining part (140) that obtains oceanic weather information by referring to the ship positional information of the operator received by the GPS reception part,
an electronic navigation chart reading module (151) that reads data matching the guide mode of the electronic navigation chart selected by the operator through the key input part or the ship positional information of the operator received by the GPS reception part from the electronic navigation chart DB,
a first screen output controlling module (152) that controls such that the read data is executed by an operating program and the executed data is displayed on a screen,
a second screen output controlling module (153) that controls such that information regarding an azimuth, a water level, a water temperature, a speed, a wind direction and a wind speed obtained by the measurement data obtaining part and position-based oceanic weather information obtained by the oceanic weather information obtaining part are obtained and the obtained information is displayed on the screen,
central control means (150) that includes a danger analyzing module (154) which obtains information regarding sudden stop and transmission of a ship to analyze whether or not the ship is abnormal and whether or not the ship meets with an accident, and
a ship information DB (160) that stores the state information and the navigation information obtained by the measurement data obtaining part.

2. The oceanic navigation system according to claim 1, further comprising:
a camera part (800) that obtains surrounding image information,
wherein the central control means (150) further includes
an image monitoring module (155) that generates a virtual boundary line on a surrounding image collected by the camera part, and recognizes an object appearing near the virtual boundary line, and
an image analyzing module (156) that analyzes a movement direction of the object recognized by the image monitoring module to recognize whether or not an abnormal situation occurs.

3. The oceanic navigation system according to claim 1, further comprising:
a magnetic compass (900) that measures an azimuth,
wherein the central control means (150) further includes an auxiliary azimuth obtaining module (157) that obtains azimuth information from the magnetic compass when it is not able to receive the azimuth information from the GPS reception part.

4. The oceanic navigation system according to claim 1,
wherein the oceanic navigation control apparatus (100) further includes a submarine topography DB (170), and
wherein the central control means (150) further includes
a submarine topography reading module (158) that reads submarine topography data matching a guide mode of the electronic navigation chart selected by the operator through the key input part or the ship positional information of the operator received from the GPS reception part from the submarine topography DB, and
a third screen output controlling module (159) that controls such that the ship positional information of the operator received by the GPS reception part maps with the read submarine topography data and the mapped data is displayed on the screen.
